(19) United States
(12) Patent Application Publication
(10) Pub. No.: US 2012/0253095 A1
(43) Pub. Date: Oct. 4, 2012

(54) CREATING A COMPENSATOR FROM SOLID PARTICULATES

(75) Inventors: John M. Wright, Dorset (GB); Michael J. Hudson, Buckinghamshire (GB)

(73) Assignee: Axellis Ventures Ltd., Market Harborough (GB)

(21) Appl. No.: 13/075,885

(22) Filed: Mar. 30, 2011

(51) Int. Cl. A61N 5/00; B22F 3/02

(52) U.S. Cl. 600/1; 419/66

(57) ABSTRACT

A patient-specific compensator is created from solid particulates on-site at a radiation treatment facility and then used there at that facility in conjunction with a radiation therapy machine to deliver radiation therapy to a cancer patient. After use, the compensator can be broken down into loose solid particulates at the facility, and another compensator can be created on-site at the facility from those particulates and used in the radiation treatment of a different cancer patient at the facility.

### TECHNICAL FIELD

The invention relates to radiation therapy and to compensators (also known as radiation filters) that are used in radiation therapy machines to provide radiation therapy to cancer patients.

### BACKGROUND INFORMATION

Intensity-modulated radiation therapy (IMRT) is a treatment method for accurately delivering a defined and uniform dose of radiation to a tumor site. This treatment method is designed to limit the amount of radiation to which peripheral non-cancerous tissues and structures are exposed. IMRT is used on cancer patients to accurately deliver a uniform dose of radiation to a patient's cancerous tissue as defined by the clinician while avoiding, or at least minimizing, radiation exposure to the surrounding healthy or critical body structures of the patient. IMRT delivers radiation to the patient's cancerous tissue from various angles and at various intensity levels in order to achieve the prescribed dose profile for that patient. Patients with cancer can be treated with other types of radiation therapy such as proton radiation therapy or cobalt radiation therapy.

With IMRT and other types of radiation therapy, the intensity of the radiation beam can be varied or modulated by using a compensator. A compensator is also known as a radiation filter. The compensator is mounted directly in the path of a radiation beam generated by a radiation therapy machine, before the beam reaches the patient. Each compensator is made specifically for a particular patient tumor and also for each angle (field) from which radiation is delivered. Existing practice utilizes compensators machined from a solid piece of material. The unique patient-specific three-dimensional geometry of each machined finished compensator provides the conformal radiation dose distributions required by that particular cancer patient to treat their tumor according to the prescribed dose. In general, a compensator created for one cancer patient cannot effectively be used for the treatment of another cancer patient. Individual compensators are used from each beam angle (field) during a course of IMRT treatment, requiring a change of compensator for each discrete field of radiation treatment. Compensators are typically provided in “sets” for a treatment plan for a specific patient. Patient-specific compensators can be machined in-house at a hospital or other radiation treatment facility, or the compensators can be ordered from a 3^{rd} party supplier such as an outside machine shop. One outside machine shop from which compensators can be ordered is .decimal, Inc of Sanford, Fla. (www.dotdecimal.com). After manufacturing the ordered compensator, the outside machine shop physically delivers that set of compensators to the requesting treatment facility, typically by shipping it to the facility using a general carrier.

### SUMMARY OF THE INVENTION

The invention generally relates to compacting solid particulates to create a compensator having certain defined and predictable radiation attenuation properties. These radiation attenuation properties may be substantially equivalent to the radiation attenuation properties of a conventional compensator produced from a solid piece of material. The solid particulates may be beads or other small pieces of one or more metals such as tungsten and/or brass, and one specific example of a suitable particulate material is crystalline tungsten powder. The solid material that the compacted particulates replace could be a solid piece of tungsten or a solid piece of brass. Empty molds can be provided to radiation treatment locations, and then, on-site at the radiation treatment facility, a mold can be filled with the particulates and those particulates compacted within the mold to form on-site a compensator for use with the specific radiation therapy machine to provide radiation treatment for the prescribed patient at that site. This avoids the need to manufacture a conventional solid-material compensator, whether that manufacturing is done at the treatment facility or remotely by an outside machine shop. Also, and again on-site at the place of treatment, a compensator formed of compacted particulates can be removed from the mold after delivery of the radiation treatment and broken down back into the individual particulates, and these loose particulates then can be placed into a different mold at the treatment facility and compacted in that different mold at the treatment facility to form a different compensator for use in the radiation treatment of a different patient at that site. The compaction is preferably performed without any added heat or added pressure and is accomplished by controlled vibration of the mold with the particulates disposed therein.

Thus, it is noted that, in summary form, the invention generally relates to creating, on-site at a radiation treatment facility, a patient-specific compensator from particulates of a radiation attenuating material that are sufficiently compacted to deliver a predictable and known attenuation to the radiation treatment, and then that compensator is used on-site at that facility in conjunction with a radiation therapy machine to deliver radiation therapy to a cancer patient. And, after use, the compacted particulates within the mold that together form the compensator can be disaggregated at the facility to recover the loose solid particulates again, and then another compensator can be created on-site at the facility from those recovered particulates and that new compensator used in the radiation treatment of a different cancer patient at the facility.

In one aspect, the invention is a method of creating a compensator on-site at a radiation treatment facility having at least one radiation therapy machine for treating cancer patients. And this method includes receiving, at the radiation treatment facility, a plurality of molds, where each of the molds is specific to a particular cancer patient and to the individual beam angles from which the cancer patient is treated. The method also includes depositing solid particulates a mold, and then compacting the solid particulates in the mold to form the compensator which is configured for use with the radiation therapy machine to treat the particular cancer patient.

In another aspect, the invention is a method of creating a compensator from solid particulates and using the compensator with a radiation therapy machine to treat a cancer patient. This method involves depositing the solid particulates into a mold and then compacting the solid particulates in the mold to form the compensator. The compensator is then placed in the path of a radiation beam generated by the radiation therapy machine during treatment of the cancer patient with the machine.

Embodiments according to either of these aspects of the invention can have various features. For example, the solid particulates can be tungsten or brass, and if tungsten the solid particulates can be crystalline tungsten powder. The solid particulates can be another material that attenuates radiation other than tungsten or brass, and the solid particulates can be combinations of two or more radiation attenuating materials. Also, the compaction can be accomplished by vibrating the mold, and without the addition of heat or external pressure. Whether accomplished by vibration or in some other way, the solid particulates can be compacted into the mold to a certain density such that the formed compensator has desired radiation attenuation properties. The compacted solid particulates can be removed from the mold, and the loose solid particulates can be recovered. The recovered particulates then can be reused with the same or another different mold to form another compensator in accordance with the invention.

Yet another aspect of the invention involves a method of creating a compensator from solid particulates where the method includes depositing the solid particulates into a mold and compacting those deposited solid particulates in the mold to form the compensator. The compensator can then be used with a radiation therapy machine to treat a cancer patient.

Objects, advantages, and details of the invention herein disclosed will become apparent through reference to the following description, the accompanying drawings, and the claims. The various disclosed embodiments as well as each of the various features of those embodiments are not mutually exclusive and can exist in various combinations and permutations whether or not expressly pointed out in the following description or the accompanying drawings.

### BRIEF DESCRIPTION OF THE DRAWINGS

In the drawings, like structures are referenced by the same or similar reference numbers throughout the various views. The illustrations in the drawings are not necessarily drawn to scale, the emphasis instead being placed generally on illustrating the principles of the invention and the disclosed embodiments.

FIG. 1A is a perspective view of one embodiment of a filling station according to the invention, with an optional tray and without a mold held in the filling station.

FIG. 1B is an exploded view showing the components of the filling station of FIG. 1A and also an empty mold.

FIG. 1C is an exploded view of the active collar of the filling station of FIGS. 1A and 1B.

FIG. 1D also is an exploded view of the active collar of the filling station of FIGS. 1A and 1B, but somewhat larger than FIG. 1C.

FIG. 2 is a cross-sectional view of a simplified version of the filling station of FIG. 1A, but with a filled mold held by the filling station.

FIG. 3 is an exploded view of a compensator assembly that includes as its main component the filled-and-compacted mold which contains compacted solid particulates and thus forms a compensator.

FIG. 4 shows a portion of a head of a radiation therapy machine with some of it in cross-section and with an indication of a radiation beam before the compensator assembly is slid into place in the path of the machine's beam.

FIG. 5A is a block diagram showing the entities involved in defining and manufacturing empty molds for use at a radiation treatment clinic for cancer patients.

FIG. 5B is flow diagram showing the steps and systems involved in creating the empty molds used at the clinic.

### DESCRIPTION

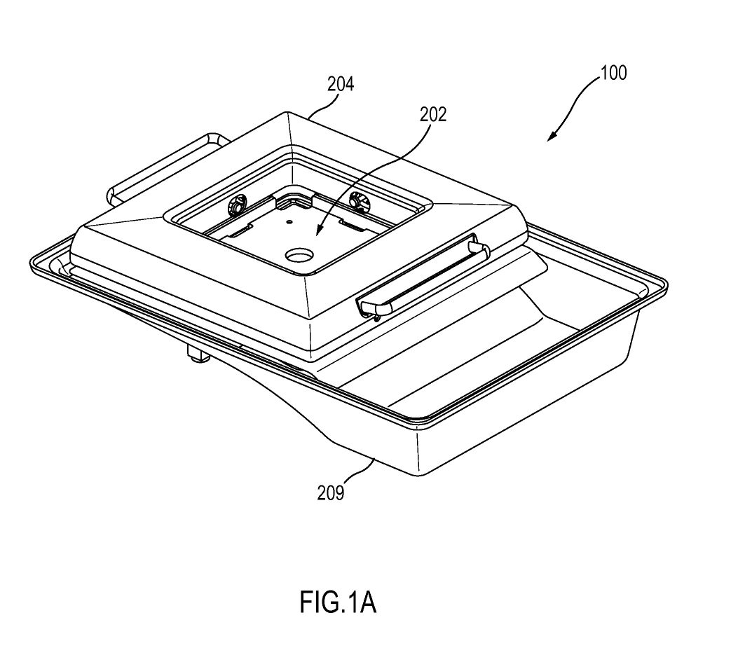

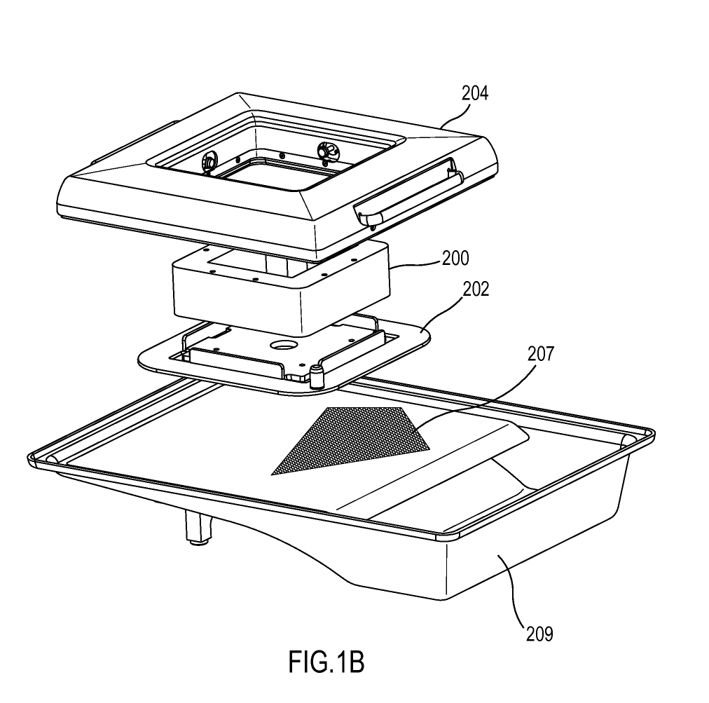

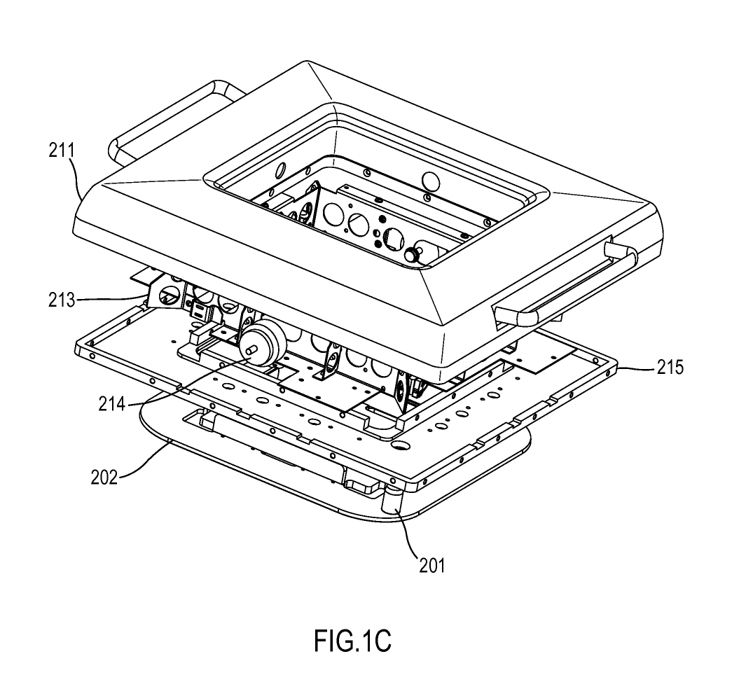

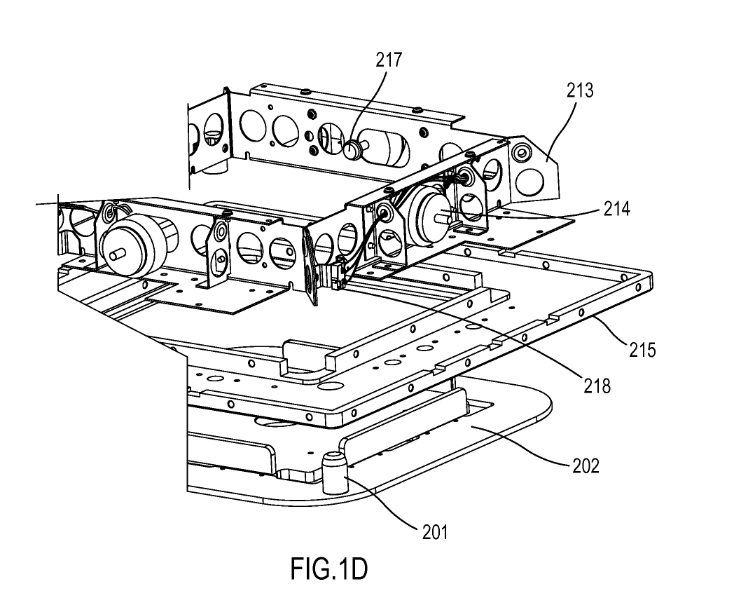

To compact, on-site at a radiation treatment facility, solid particulates into a patient-specific mold such that the resulting compensator (which comprises the mold with the particulates compacted therein) has predetermined, and predictable and consistent, radiation attenuation properties, an apparatus must be present at the facility. The apparatus can be referred to as a filling station, and various details and functionality of the filling station are described herein. FIGS. 1A, 1B, 1C, and 1D show one embodiment of a filling station 100 that can be used on-site at a radiation treatment facility in accordance with the invention.

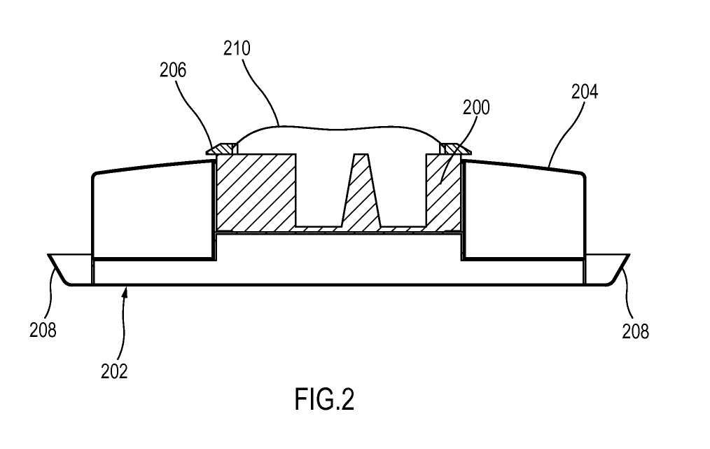

Referring specifically now to FIGS. 1A and 1B, the disclosed filling station 100 includes a support plate 202, an active collar 204, and an optional tray or reservoir 209. The reservoir 209 is designed to hold the solid particulates (such as crystalline tungsten) and to keep a clean working environment. The reservoir 209 is similar in its configuration to a large paint roller tray. A platform made of a fine mesh 207 is inside the reservoir 209. The mesh platform 207 cleans any foreign matter out of the solid particulates, and the mesh platform 207 also allows any spillage when filling a mold 200 to be kept away from the mold 200 and the active collar 204. The support plate 202 is disposed on the mesh platform 207. The support plate 202 has a pair of feet (not shown) that stop the support plate 202 from moving around on the surface of the mesh platform 207. The support plate 202 has shallow walls which provide a positive location for the mold 200 to sit and stay when the mold 200 is placed on the support plate 202. The support plate 202 also has a pair of locating pins or dowels 201 on its upper surface which locate the active collar 204 and also trip a micro switch 218 that enables the active collar 204 to be switched on. It is possible to use one dowel 201 or more than the disclosed two dowels 201. The use of the micro switch 218 ensures that if the active collar 204 is not placed onto the support plate 202 then the active collar 204 will not switch on and thus will not vibrate. After locating the mold 200 onto the support plate 202, the active collar 204 is slid over the mold 200 to locate the active collar 204 on the dowels 201. Once the active collar 204 is in position, a retaining ring 206 (FIG. 2) can be placed onto the periphery of the mold 200.

The empty mold 200 can be half filled with the solid particulates tungsten and the active collar 204 can be switched on. Once the active collar 204 is energized, the mold 200 will be filled so that the level of particulates forms a mound limited by the retaining ring. The active collar 204 will operate for a given amount of time according to a time value associated with the particular mold 200 held by the filling station 100, and this value can be stamp onto or printed on a label of the mold 200, for example. When the time has elapsed, the active collar 204 stops vibrating, and the retaining ring 206 can be removed. The active collar 204 then can be lifted off and removed from about the now-filled and compacted mold. The particulates on the top surface of the filled/compacted mold can then be carefully scraped level to the top face of the mold using a straight edge. The surplus particulates that are scraped off onto the mesh platform 207 where those scraped-off particulates are filtered through the mesh and returned to the reservoir 209. The scraped-off particulates that are returned to the reservoir 209 are available for use to fill and compact the next empty mold at the filling station 100.

The active collar 204 is called such (that is, “active”) because it contains the moving parts that impart the energy to the mold and thus to the particulates deposited within the mold. The energy that the active collar 204 imparts to the mold, and also to the particulates that the mold contains, is what causes the particulates to become compacted into the mold to form a radiation-attenuating compensator. The disclosed filling station 100 uses the active collar 204 shown in further detail in FIGS. 1C and 1D. Four spring loaded solenoids 214 are employed within the active collar 204, although it is noted that more or less than four solenoids could be used such as one, two, three, five, etc. The solenoids 214 impact directly onto the sides of the mold 200 when the mold 200 is placed on the support plate 202 and the active collar 204 is placed on/around the mold 200. More accurately stated, each solenoid 214 has an anvil 217 that faces in toward where the mold 200 will be placed in the filling station 100, and it is the anvil(s) 217 that actually impact the side(s) of the mold 200 to vibrate the mold and thus compact the particulates deposited within the mold. This design, where each solenoid 214 has an anvil 217 that contacts the mold directly, is relatively simple and cost-effective to build and use. The disclosed design of the active collar 204 uses four solenoids 214, each of which has an associated anvil 217.

As shown in FIGS. 1C and 1D, the active collar 204 includes a base plate 215, a cover 211, and a chassis 213. Not shown is an electronic control module, but the active collar 204 would have one in order to control the solenoid(s) 214. The chassis 213 holds the solenoid(s) 214 and their anvil(s) 217. The chassis 213 locates the solenoid(s) 214 and provides a robust frame from which the solenoid(s) 214 hang. The forces imparted by the solenoid(s) 214 are not high, typically less than 10 lb force over a 0.2 inch movement, but the chassis 213 should be structurally robust enough to prevent flexing that would absorb too much of the energy created by the solenoid(s) 214. Mounted to the chassis 213 is a micro switch 218 which ensures the collar 204 cannot be energized unless it is placed over the support plate 202. This is a safety feature and an operational feature, as damage could be imparted to the mold 200, and/or to the active collar 204, if the collar is placed over the mold 200 while the impacting anvils 217 are active.

Another arrangement of solenoids within the active collar 204 could cause vibration of the mold as follows. With the solenoids in their “free state” (that is, no power supplied to the active collar), each solenoid's return spring will pull the anvil away from the mold's side surface and at the same time return the solenoid to the in-active position. On the application of the electrical current, each of the energized solenoids pulls the solenoid bobbin (the moving part of the solenoid) into the solenoid coil with sufficient force to cause the anvil to impact the outer surface of one of the sides of the mold, and this imparts the energy (in the form of vibration) to compact the particulates disposed within the mold's cavity. As the solenoid is operating on a lever, the movement of the solenoid is multiplied by the ratio of the solenoid-to-pivot versus the anvil-to-pivot distance. This distance results in a shorter travel of the solenoid and hence a higher frequency is possible. The resulting higher frequency causes more vibration and increased energy in the particles within the mold. In addition, the ratio increases the impact force by the same ratio.

Still other ways of imparting the necessary energy to a mold are possible to achieve the desired level of compaction of particulates within the mold. For example, the support plate 202 could be mounted upon a motor connected to eccentrically weighted shaft by means of a toothed belt, smooth belt, or gear train. The eccentrically loaded shaft could impart vibration to the base of the mold 200 through the support plate 202, and, if the shaft comprised a cam, the cam could impact on a plunger to tap on the support plate 202 imparting direct energy into the mold 200. Further, the support plate 202 could house an off-the-shelf vibrating solenoid assembly (such as those available from Kedrion Tri-Tech, LLCF of Mishawaka, Ind. which has a web site at www.kendrion-tritech.com) similar to those found on industrial vibrating component feeders (such as those available from Automation Devices, Inc. of Fairview, Pa. which has a web site at www.autodev.com) or vibrating component finishing devices, wherein the solenoid assembly oscillates at the frequency of the supplied electrical current, or a modified input frequency, whereby the off-the-shelf vibrating solenoid directly imparts its vibration to the support plate 202.

One version of the disclosed embodiment of the filling station 100 weighs less than 20 kg. With a filled mold at the filling station 100, the total weight of the filling station 100 is no more than 50 kg, or else 80 kg of total weight if 30 kg of crystalline tungsten powder is provided within a covered receptacle of the filling station 100. The size of the filling station 100 is such that it fits on a standard office or laboratory desk, and it can be configured to accommodate a compensator that is at least 295 mm by 235 mm.

The filling station 100 is capable of compacting solid particulates to a density of at least 10.15 g/cc±0.05 g/cc, and as a specific example to a density of 11 g/cc, where g/cc stands for gram per cubic centimeter. This density is sufficient to create a compensator with predictable and consistent radiation attenuation properties, such as radiation attenuation properties that are substantially the same as, or at least somewhat close to, those of a conventional compensator formed from a solid piece of material. A solid tungsten compensator, for example, could have a density of 19.3 g/cc. A mold that defines a particular patient-specific compensator shape is used at the filling station. Solid particulates, such as crystalline tungsten powder of grade C120 which is available from Buffalo Tungsten Inc. of Depew, N.Y. (www.buffalotungsten.com), are deposited into the mold at the filling station. The filling station imparts vibration to the mold and thus the solid particulates disposed within the mold. The frequency of vibration provided to the mold by the filling station is 75 Hz or higher, but the vibration frequency is not high enough to interfere with any standard office or personal equipment such as wireless communication systems, computer screens, or wired communication systems. The vibration is applied to the filled mold for 10 minutes or less in order to create the compensator. In less than 10 minutes, the filling station 100 can create a compensator with a density of at least 10.15 g/cc±0.05 g/cc and with a weight of 2 kg to 17.5 kg.

Referring to FIG. 2, an empty mold 200 is placed on the support plate 202 of the filling station 100, and the active collar 204 is placed around the mold 200. The collar 204 fits onto the plate 202 as shown, and together the plate 202 and the collar 204 prevent the mold 200 from moving or falling off or out of the filling station 100 when vibration is applied to the mold 200 by the filling station 100. Outer-edge cut-out portions 208 beyond the collar 204 can be provided, as shown, and these portions 208 are designed to receive any excess particulates 210 that might fall out of the mold 200 when the filling station 100 vibrates the mold 200. The portions 208 thus provide the same purpose as the tray or reservoir 209 described previously with regard to FIGS. 1A and 1B. A retaining ring 206 is placed on the top surface of the mold 200 as shown. The retaining ring 206 allows the solid particulates 210 that are deposited into the mold 200 (such as, for example, crystalline tungsten powder) to be built up in a mound above the mold 200 as shown. Mounding the solid particulates 210 in this manner provides a volume of solid particulates 210 sufficient to fill any voids within the mold 200 that might be exposed during vibration. The mounding of the particulates 210 into and on the mold 200 also adds weight that helps to increase compaction of the particulates 210 in the mold 200. Once the mold 200, collar 204, and ring 206 are in place on the plate 202, the filling station 100 can be switched on to provide vibration to the mold 200. The vibration is applied by the filling station 100 for a period of time determined by the amount and type of particulates 210 deposited into/on the mold 200. After sufficient compaction of the particulates 210 into the mold is achieved by virtue of the vibration being applied by the filling station 100 for a certain amount of time, the filling station 100 is switched off to stop the vibration, and then the retaining ring 206 is removed. The collar 204 is then removed as well. The top surface of the filled/compacted mold 200 is then carefully scraped level with a straight edge, and the compensator thus is completed and ready to be removed from the plate 202 and used in a compensator assembly 300. The compensator assembly 300 is designed to be inserted into a radiation therapy machine for use therewith during the radiation treatment of a cancer patient on-site at the treatment facility where the filling station 100 is located and where the compensator was created with the filling station 100. The mold with the compacted particulates could weigh nearly 50 lbs., and thus it would be difficult to lift such a heavy item out of a hole or opening in the plate 202, and that is why the plate 202 is configured as shown. It is much easier to slide a heavy compacted mold off of the plate 202 than it would be to lift the heavy compacted mold out of a hole.

It is noted that the filling station 100 is not heated and does not add heat during the process of creating the finished compensator 312. The filling station 100 also does not exert any extra pressure down onto the particulates disposed in the mold 200. The only pressure or force applied to the particulates within the mold 200 is due to the weight of the collective particulates disposed within the mold 200. The necessary compaction of the particulates in the mold 200 to create the finished compensator 312 comes from just the vibration applied to the mold 200 by the filling station 100 and also the weight of the particulates themselves as they push down into the mold 200 due to the weight of the particulates themselves within the mold 200 and mounded on top of the mold 200.

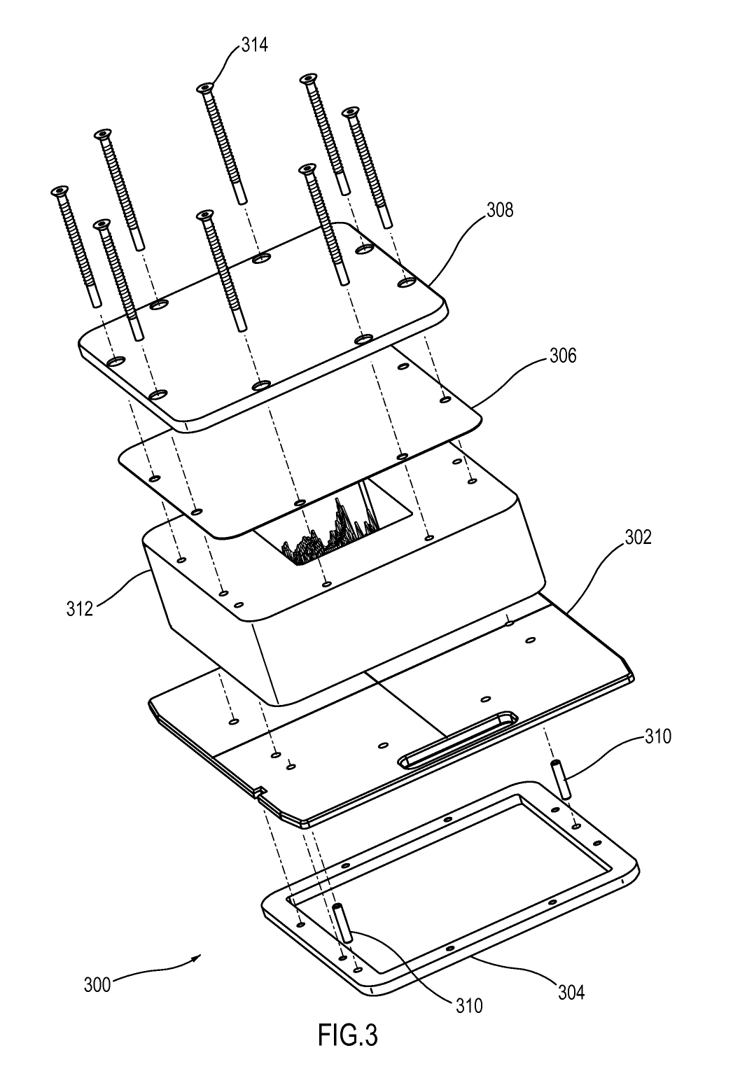

The finished compensator can be taken from the plate 202 and assembled together with other components on-site at the treatment facility to form a compensator assembly 300. Referring to FIG. 3, the compensator assembly 300 is created by combining it with an accessory block which is also referred to as an accessory tray 302. This tray 302 is basically a base which allows the compensator assembly 300 to be inserted and received into a radiation therapy machine located at the treatment facility. To form the assembly 300, the finished compensator 312 is sandwiched between the tray 302 and a clamp 304, on one side (bottom), and a gasket 306 and a top cap 308, on its other side (top). The clamp 304 can be formed of aluminum, and it can use two or more dowels 310 that extend through the tray 302 and into the bottom of the finished compensator 312. The gasket 306 can be made of polypropylene, and the top cap 308 can be made of polycarbonate. Multiple clamp screws 314 (such as eight of them, as shown) can be used to hold the assembly 300 together. The finished compensator 312 is made at the filling station 100 as described herein, and the finished compensator 312 includes the mold 200 as well as the compacted particulates within the mold 200. Once completed, the compensator assembly 300 is processed through a clinical quality assurance (QA) process at the treatment facility before it is mounted within a radiation therapy machine at the facility and used to treat a cancer patient at the facility.

It is noted that the mold 200 typically will have on one or more of its four lateral sides (not its bottom, and not its top which is open to receive the solid particulates) one or more markings, or one or more labels with one or more markings, that uniquely identify the mold 200 as belonging to a particular patient. Every patient must have a unique set of compensators created for that particular patient's specific radiation treatment requirements, and that is why each mold 200 must be able to be tracked and uniquely identified at all times including when it is at the filling station 200 being turned into a finished compensator 312 and when it is part of a compensator assembly 300.

It also is noted that a mold 200 can be machined in a low density polyurethane material. This material can be referred to as foam, but it actually is, at least in one embodiment according to the invention, a rigid polymer based material with a particular density. The density can be, as one example, 10 lb/cu. Ft. This particular exemplary density allows for reasonable ease in machinability, speed of machining, reliability of feature definition, and transparency to radiation. These characteristics (that is, machinability ease and speed, reliable and accurate surface feature definition, and transparency to the radiation therapy machine's radiation beam) typically are the most important characteristics to consider and account for when selecting the material to use to form the mold 200. After processing data generated by a Treatment Planning System (TPS) at the clinic, the inner surface and contours (that is, the cavity) of the mold 200 can be calculated. The mold design takes the form of a tessellated surface from which the CNC (computer numerical control) tool path is calculated. In addition, the unique inspection routine is prepared using the surface form to define the points. The mold can then be machined and inspected on the same CNC machine tool. When each of the molds 200 that together comprise the set of molds needed to treat a particular cancer patient has been completed and has passed inspection, that complete set of molds 200 for that particular patient's radiation treatment procedure can be delivered to the clinic where each of the molds 200 can then be filled with solid particulates and vibrated on-site at the clinic by using a filling station 100 to create the unique set of finished compensators 312 that are needed to deliver the radiation treatment at the clinic to that particular cancer patient using a radiation therapy machine located at the clinic, all as described herein.

This business model of shipping just a set of patient-specific molds 200 to the clinics (and not completed compensators), where the molds 200 are then filled and compacted on-site at the clinics to create the needed set of completed patient-specific compensators on-site at the clinics, allows a box of, for example, ten molds, which typically will weigh less than 10 lbs, to be shipped from the location where the molds 200 are created to the location of a clinic. A single conventional compensator formed of a solid piece of brass, for example, typically weighs in excess of 10 lbs. It thus is much less expensive and much more convenient to ship just the molds 200 to the treatment clinics, and not the heavy completed compensators as is typically done. The freight costs to ship a plurality of molds 200 typically will be much lower than shipping even a single conventional compensator.

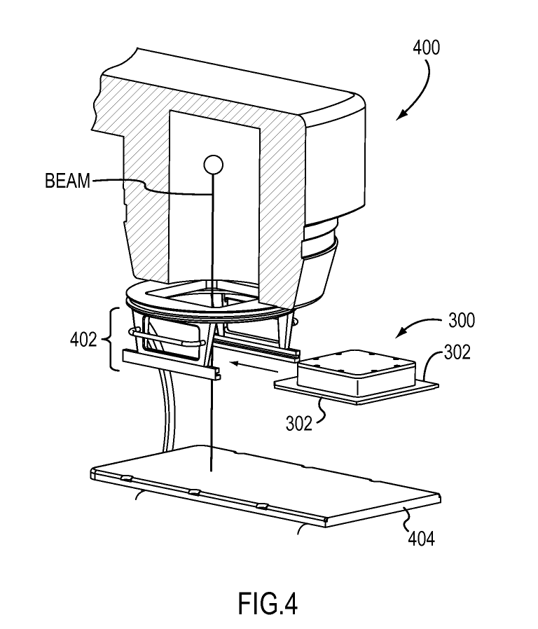

Referring now to FIG. 4, it can be seen how the compensator assembly 300 is mounted within a head of a radiation therapy machine 400 by sliding the assembly 300 into a frame of the head of the machine 400 that is called the accessory mount 402. Two sides of the tray 302 are received by cooperating slots or tracks of the accessory mount 402. The compensator assembly 300 can be inserted into the mount 402 in the orientation shown where the machine's radiation beam first hits and passes through the top of the finished compensator 312, or the compensator assembly 300 can be inserted below the accessory tray 302. The finished compensator 312 or the compensator assembly 300 may also be mounted in a slot immediately below the head of the linear accelerator referred to as a Wedge Tray slot. It is noted that, when the compensator assembly 300 is mounted in place to the head of the radiation therapy machine 400, the machine's head typically is rotated to certain distinct positions where the head then stops to allow the machine 400 to deliver the radiation beam to the cancer patient at that position. The amount of radiation delivered by the radiation beam in any particular position of the head is determined in advance by the TPS. Each time the head of the machine 400 is moved to a new position to deliver radiation to a cancer patient during a treatment session for that patient, the compensator assembly 300 can be, and typically is, changed to mount to the machine 400 a compensator assembly having a specific compensator 312 designed specifically to adjust the beam's radiation intensity for that particular head location for that particular patient during the radiation treatment. In other words, each series of beam sequences delivered to a particular patient during a treatment procedure for that patient typically requires a different and distinct finished compensator 312.

Also, and with continued reference to FIG. 4, it is noted that, prior to treating any patient with radiation, the staff at the hospital, clinic, or other radiation treatment facility are required to check that each beam delivers the radiation as planned by the TPS. To do this, a sensitive medium of either film or an electronic dose measurement device is placed on the treatment couch 404, and then each beam is delivered and measurements are taken with the proper compensator assembly 300 mounted into the radiation therapy machine 400 at each beam delivery location of the machine's head for the whole treatment procedure for a particular patient. The measured radiation delivery is then compared to the delivery calculated by the TPS, and only if the match between the actual and calculated delivery exceeds a threshold used at the clinic (normally in excess of 80%) is the treatment considered viable.

After the treatment program is completed for a particular cancer patient, and if one or more of the finished compensators 312 used in that patient's treatment procedure are no longer needed for a radiation treatment procedure, the compensator assembly 300 can be broken down into its component parts shown in FIG. 3. The finished compensator 312 then can be emptied of its compacted particulates, and those compacted particulates released from the mold 200 can be broken down (that is, disaggregated) into their prior state of loose solid particulates. These recovered particulates then can be reused in the same, or more typically a different, mold 200 to form a different finished compensator 312.

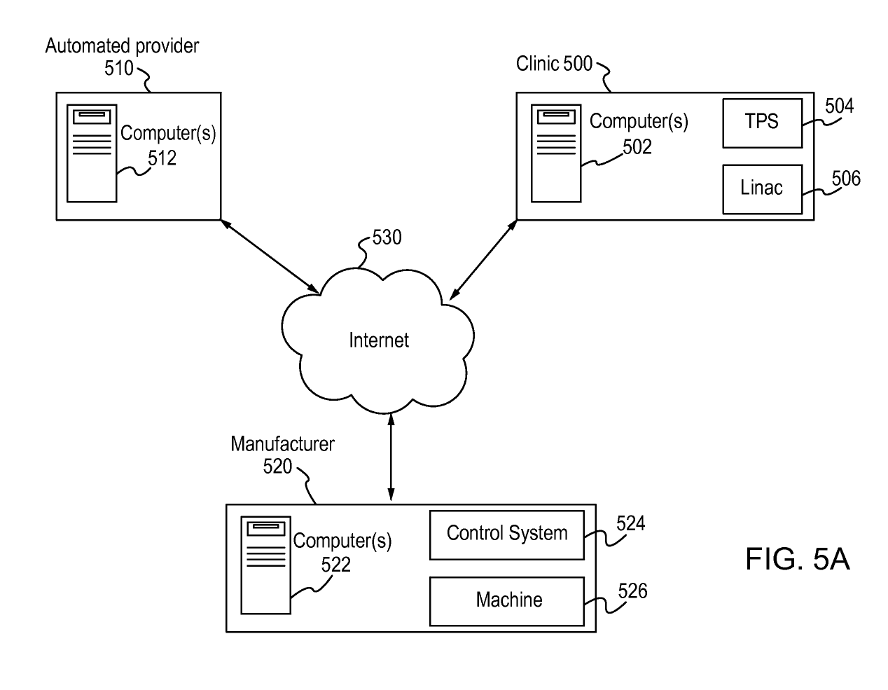

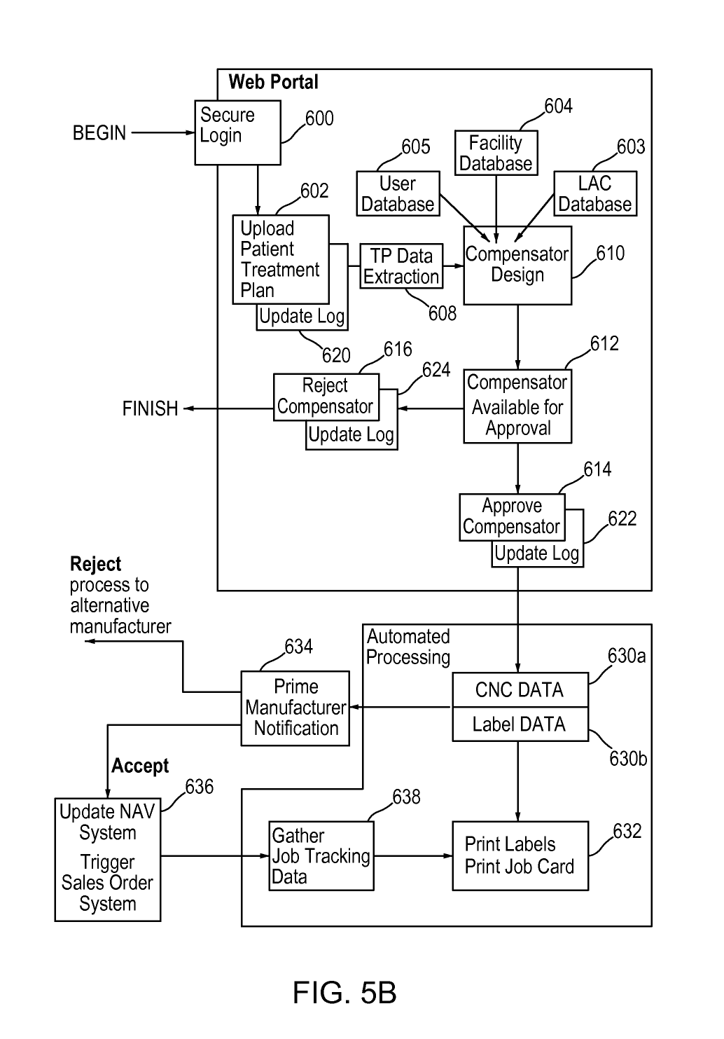

It has been described herein how a set of patient-specific molds 200 is physically created at a manufacturing site and then shipped (by using, for example, a delivery service such as one offered by United Parcel Service of America, Inc. of Atlanta, Ga. or one offered by FedEx Corporation of Memphis, Tenn.) from the manufacturing site to a clinic where those molds 200 are then filled and compacted on-site at the clinic to create the needed set of completed patient-specific compensators 312 to be used with a radiation therapy machine 400 at the clinic to treat a particular patient. How each patient-specific mold 200 is created will now be described in further detail, with reference to FIGS. 5A and 5B.

As shown in FIG. 5A, the entities involved in defining and manufacturing molds for use at a radiation treatment clinic include the clinic 500, an outside provider 510, and a manufacturer 520 such as a machine shop. The clinic 500 can be referred to as a radiation oncology treatment center, and it can be a hospital or any location where cancer patients are treated with radiation to address their cancerous tumors. Each clinic 500 typically will have at least one radiation therapy machine 400 such as a linear accelerator 506 and also at least one supporting TPS 504. The linear accelerator 506 is also referred to as a “Linac”. Linacs are available commercially from Varian Medical Systems, Inc. (of Palo Alto, Calif.) and also from Siemens Medical Solutions USA, Inc. (of Malvern, Pa.), for example. The TPS 504 is available commercially from Varian under the name “Eclipse” and also from other vendors which use other product names for their treatment planning systems. The clinic 500 typically also will have at least one computer 502 such as a general purpose desktop computer with the typical components including at least one processor, memory (such as RAM and/or ROM), one or more other storage mechanisms or devices (hard drive, for example), at least one display screen, one or more input devices such as a keyboard and/or a mouse, a web browser application such as “Internet Explorer” by Microsoft Corporation of Redmond, Wash., and in general the ability to store instructions in the memory and/or storage devices (more generally, computer-readable media) that are executed by the processor(s) to cause the processor, and thus the computer 502, to perform various functions such as web browser functions as well as other functions. A user of the computer 502 can access the World Wide Web via the Internet 530 by launching and using the computer's web browser. Someone at the clinic 500 also could use the TPS 504 to access the Web via the Internet 530 by launching and using a web browser that the TPS 504 might have. As is typical, the Internet 530 is represented in FIG. 5A as a “cloud” which is a metaphor for the Internet. The cloud 530 can be any type of computer or communications network but in the disclosed embodiment is the Internet. Someone at the clinic 500 can use the computer 502 to communicate over the network 530 with one or more server computers 512 located at the outside provider 510. The computer(s) 512 can be one or more web servers. With the computer(s) 512, the provider 510 can communicate via the network 530 with the clinic 500 and also with the manufacturer 520. (While the one or more servers 512 are shown located at the provider 510, it is noted that they do not have to be physically at the same geographic location of the provider 510, and instead the servers 512 could be located at some location remote from the provider 510. That is, at least some of the services and functions performed by the provider 510 could be outsourced or hosted on one or more servers 512 located remote from the business address of the provider 510.) The manufacturer 520 includes at least one computer 522 and equipment to create one or more physical molds 200. The mold-creating equipment located at the manufacturer 520 can include at least one machine control system 524 and at least one controllable machine 526 such as a CNC machine. Once physical molds 200 are created at the manufacturer 520, those molds 200 can be shipped from the manufacturer 520 to the clinic 500 where the molds 200 then can be filled/compacted with solid particulates on-site at the clinic 500 according to the invention and to create the finished compensators 312 needed for use with the Linac 506 or other radiation therapy machine 400 to treat one or more cancer patients at the clinic 500.

FIG. 5B shows steps and systems involved in creating the molds 200 that are used at the clinic 500. Someone at the clinic 500 (such as a technician or a doctor or other clinician) uses the TPS 504 to create a patient treatment plan for a specific cancer patient and also to create one or more corresponding computer files for that plan. Someone at the clinic 500 then can use the computer 502 (or, alternatively, the TPS 504 which can be a computer similar to the computer 502, but the TPS 504 also will have and run one or more specialized applications that allow it to perform all of the functions of a TPS) to login over the Internet 530 to the server(s) 512 of the provider 510 (step 600) and transmit the treatment plan file(s) to server(s) 512 by, for example, uploading them from the computer 502 to the server(s) 512 over the Internet 530 (step 602). The server 512 then processes the uploaded file to create a compensator mold design 610. The processing done by the server(s) 512 to create the mold design 610 includes considering radiation transmission data (available from a Linac database 603), patient data (available from a user database 604), and facility data (available from a facility database 605), and also includes extracting the number of radiation beams and beam angles (step 608). Someone at the clinic 500 then can access the server(s) 512 (using the computer 502 or the TPS 504, and via the Internet 530) to review the design 610 (step 612) and either approve (step 614) or reject (step 616) the design 610. If approved, the server 512 then processes the approved mold design 610 into CNC cutting and CNC inspection instructions 630a for use at a manufacturer 520 to create and inspect each unique mold 200. This processing by the server 512 includes processing each beam individually to create three-dimensional mathematical models of the mold 200 to allow instructions to be generated that indicate how to cut and create the mold 200 on one or more machines at the manufacturer 520. The server 512 also creates various other information that can be sent to the manufacturer 520 regarding the approved mold design 610 such as a purchase order for the manufacturer 520 and data to go on a label for each unique mold 630b. The server 512 can print certain identifying information for each unique mold to allow that printed information to be physically filed and stored as paper records (step 632). The server 512 collects and maintains in storage detailed logs of all uploaded treatment plan files, design approvals, and design rejections (steps 620, 622, and 624), and these logs can be saved in permanent storage (such as on a computer hard drive, tape, or compact disc) by the server 512 for a certain period of time such as seven years. The server 512 can send a notification to the manufacturer 520 (such as by email) to verify that the manufacturer 520 will accept the mold-creation order (step 634). If the order is accepted and verified by the manufacturer 520, the server 512 can send electronically over the Internet 530 to the manufacturer 520 all order instructions and related information to allow the manufacturer 520 to create the physical mold(s) 200 defined by the instructions/information (step 636). The server 512 also can collect from the manufacturer 520 data about the accepted/verified order and its status (step 638).

With continued reference to FIG. 5B, it is noted that the label data 630b can be in the form of a barcode. The labels with the barcodes can be created by the server(s) 512 and sent electronically to the computer(s) 522 of the manufacturer 520 where each of the physical labels can be printed and applied to the appropriate mold. Or the labels could be printed elsewhere and shipped to the manufacturer 520. In any event, each mold design can have assigned to it a globally unique identification (GUID) number that goes along with that design throughout its lifecycle from design to creation of the mold and then creation of the powder-filled compensator from that mold. The physical mold can have applied to it a label with the GUID number in the form of a barcode. It is not required to use a barcode to capture or represent the GUID number, but a barcode is useful because barcode readers are available and well known. With each unique mold and compensator bearing a GUID number in the form of a barcode, all parties involved (such as clinicians, machinists, etc.) are able to track and identify specifically each individual mold design, mold, and compensator. More particularly, it is noted that each barcode is generated from the GUID number assigned to the compensator at its design stage from the compensator-surface modeling once clinically approved and the order placed. This GUID number is assigned to the CNC programs and the CNC inspection routines required for the production of a particular compensator mold. The barcode is attached to the compensator mold blank prior to machining at the manufacturer to create the mold. A barcode reader will be on or associated with the CNC machine at the manufacturer. A probing system will check to make sure that the compensator mold blank is loaded correctly, and then the barcode reader is called to read the label on the blank to cause the CNC control system 524 to pull the correct program and inspection routine from the server(s) 512 or the computer(s) 522. The ultimate goal of using GUID numbers (in, for example, the form of barcodes on labels) is to reduce risk at the point of radiation delivery to a cancer patient. When full automation is employed, a compensator is loaded into the Linac 506 and a barcode reader automatically scans the label in-situ, thereby causing the Linac 506 to rotate to the appropriate beam angle and set the number of monitor units (MUs) for delivery to the patient. Ideally, this will only happen once per treatment program unless manually overridden, and if the wrong compensator were placed in the wrong field/beam angle, then the Linac would not deliver its dose. In order to achieve such full automation, the maker of the Linac likely will have to cooperate by providing an appropriate interface to the Linac's control systems.

Certain embodiments according to the invention have been disclosed. These embodiments are illustrative of, and not limiting on, the invention. Other embodiments, as well as various modifications and combinations of the disclosed embodiments, are possible and within the scope of the disclosure.

What is claimed is:

1. A method of creating a compensator on-site at a radiation treatment facility having at least one radiation therapy machine for treating cancer patients, comprising:
receiving at the radiation treatment facility a plurality of molds, each of the molds being specific to a particular cancer patient;
depositing solid particulates into one of the molds; and
compacting the solid particulates in the mold to form the compensator which is configured for use with the at least one radiation therapy machine to treat the particular cancer patient.

2. The method of claim 1 wherein the depositing step comprises depositing solid particulates of tungsten or brass into one of the molds.

3. The method of claim 1 wherein the depositing step comprises depositing crystalline tungsten powder into one of the molds.

4. The method of claim 1 wherein the compacting step includes vibrating the mold.

5. The method of claim 4 wherein the compacting step does not include adding heat.

6. The method of claim 1 wherein the compacting step comprises compacting the solid particulates in the mold to a predetermined density.

7. The method of claim 1 wherein the compensator that is formed by the compacting step includes the mold.

8. The method of claim 1 further comprising placing the compensator in the path of a beam generated by the machine during treatment of the patient with the machine.

9. The method of claim 1 further comprising removing the compacted solid particulates from the mold and recovering the solid particulates.

10. A method of creating a compensator from solid particulates and using the compensator with a radiation therapy machine to treat a cancer patient, comprising:
depositing the solid particulates into a mold;
compacting the solid particulates in the mold to form the compensator; and
placing the compensator in the path of a radiation beam generated by the radiation therapy machine during treatment of the cancer patient with the machine.

11. The method of claim 10 wherein the depositing step comprises depositing solid particulates of tungsten or brass into the mold.

12. The method of claim 10 wherein the depositing step comprises depositing crystalline tungsten powder into the mold.

13. The method of claim 10 wherein the compacting step includes vibrating the mold.

14. The method of claim 13 wherein the compacting step does not include adding heat.

15. The method of claim 10 wherein the compacting step comprises compacting the solid particulates in the mold to a predetermined density.

16. The method of claim 10 wherein the compensator that is formed by the compacting step and used in the placing step includes the mold.

17. The method of claim 10 wherein the placing step comprises mounting the compensator to the machine to place the compensator in the path of the beam generated by the machine during the treatment of the cancer patient with the machine.

18. The method of claim 10 further comprising removing the compacted solid particulates from the mold and recovering the solid particulates.

19. A method of creating a compensator from solid particulates, comprising:
depositing the solid particulates into a mold; and
compacting the solid particulates in the mold to form the compensator which is configured for use with a radiation therapy machine to treat a cancer patient.

20. A method of using solid particulates to create a compensator for use with a radiation therapy machine to treat a cancer patient, comprising:
depositing the solid particulates into a mold; and
compacting the solid particulates in the mold to form the compensator.
